(19) United States
(12) Patent Application Publication
(10) Pub. No.: US 2024/0117133 A1
(43) Pub. Date: Apr. 11, 2024

(54) Polymer Films

(71) Applicants: Fujifilm Manufacturing Europe B.V., Tilburg (NL); Fujifilm Corporation, Tokyo (JP)

(72) Inventors: Jacko Hessing, Tilburg (NL); Elisa Huerta Martinez, Tilburg (NL); Adrianus Jacobus Van Rijen, Tilburg (NL); Renée Maria Ripken, Tilburg (NL); Takeshi Narita, Tilburg (NL)

(21) Appl. No.: 18/271,547

(22) PCT Filed: Jan. 27, 2022
(86) PCT No.: PCT/EP2022/051934

§ 371 Date: Jul. 10, 2023

(30) Foreign Application Priority Data
Jan. 28, 2021 (GB) 2101153.1

(51) Int. Cl. C08J 5/22; B01D 67/00; B01D 69/02; B01D 71/66; B01J 39/05; B01J 39/19; B01J 39/20; B01J 47/12; C02F 1/42; C02F 1/469; C08F 212/34; C08F 212/36; C08G 75/045; C09D 4/00; C09D 125/18; C09D 181/02; H01M 8/103; H01M 8/1032; H01M 8/22

(52) U.S. Cl. CPC C08J 5/2275; B01D 67/0006; B01D 69/02; B01D 71/69; B01J 39/05; B01J 39/19; B01J 39/20; B01J 47/12; C02F 1/42; C02F 1/4693; C08F 212/34; C08F 212/36; C08G 75/045; C09D 4/00; C09D 125/18; C09D 181/02; H01M 8/103; H01M 8/1032; H01M 8/227; B01D 2323/30; B01D 2323/345; B01D 2325/42; C02F 2001/425; C08F 2800/20; C08J 2325/18; C08J 2381/02; H01M 2008/1095

(57) ABSTRACT

A polymer film comprising anionic groups and 0.008 to 25 mg/g of each of components (a) and (b): (a) a crosslinking agent which is free from fluoro groups and comprises a group of Formula (I); and (b) a non-ionic crosslinking agent; Formula (I) wherein M⁺ is a cation and * indicates the attachment points to other elements of the crosslinking agent.

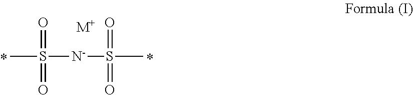

The present invention relates to polymer films, to compositions suitable for making polymer films, to cation exchange membranes and to their preparation and use.

Ion exchange membranes are used in electrodialysis, reverse electrodialysis, electrolysis, diffusion dialysis and a number of other processes. Typically the transport of ions through the membranes occurs under the influence of a driving force such as an ion concentration gradient or, alternatively, an electrical potential gradient.

Ion exchange membranes are generally categorized as cation exchange membranes or anion exchange membranes, depending on their predominant charge. Cation exchange membranes comprise negatively charged groups that allow the passage of cations but reject anions, while anion exchange membranes comprise positively charged groups that allow the passage of anions but reject cations. Some ion exchange membranes comprise a porous support which provides mechanical strength. Such membranes are often called “composite membranes” due to the presence of both an ionically-charged polymer which discriminates between oppositely charged ions and the porous support which provides mechanical strength.

Cation exchange membranes may be used for the treatment of aqueous solutions and other polar liquids, and for the generation of electricity.

Electricity may be generated using reverse electrodialysis (RED) in which process standard ion exchange membranes may be used. Cation exchange membranes may also be used for the generation of hydrogen, e.g. in fuel cells and batteries.

According to a first aspect of the present invention there is provided a polymer film comprising anionic groups and 0.008 to 25 mg/g of each of components (a) and (b):
(a) a crosslinking agent which is free from fluoro groups and comprises a group of Formula (I); and
(b) a non-ionic crosslinking agent:

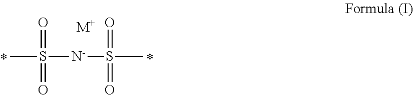

wherein M⁺ is a cation and * indicates the attachment points to other elements of the crosslinking agent.

For the avoidance of doubt, the anionic groups of the polymer film are covalently bound, i.e. form part of the polymer structure, and are not free electrolytes.

The amounts of components (a) and (b) are expressed relative to the weight of the polymer film (in mg/g or promille).

Preferably the polymer film comprises 0.015 to 20 mg/g, especially 1 mg/g to 15 mg per gram polymer film, of component (a).

Preferably the polymer film comprises 0.015 to 20 mg/g, especially 1 mg/g to 15 mg per gram polymer film, of component (b).

The amount of component (a) present in the polymer film may be the same as or different to the amount of component (b) present in the polymer film.

The polymer film optionally contains one or more than one component (a) and/or one or more than one component (b). When the polymer film contains more than one component (a) or (b) the above-stated preferred amounts of component (a) or (b) relate to the total amount of component (a) or (b).

A content above 25 mg/g (of (a) or (b)) is indicative for an insufficiently cured polymer film and a content below 0.008 mg/g (of (a) or (b)) is indicative of an excessively cured polymer film. The amounts of components (a) and (b) specified herein provide a polymer film with particularly good permselectivity, especially under strong basic conditions.

The desired amount of components (a) and (b) may be included in the polymer film of the present invention by preparing the polymer film from a composition comprising components (a) and (b) and partially but not completely curing the components (a) and (b) to form the polymer film. In this way some of component (a) and component (b) remain in the polymer film.

The presence and amount of components (a) and (b) in the polymer film may be determined by optionally drying the film, placing the (dry) film in a solvent or a mixture of solvents suitable for dissolving the component (a) and/or (b) (e.g. water, ethanol, isopropyl alcohol) for (at least) 16 hours and then determining the presence and amount of components (a) and (b) in the solvent or mixture of solvents using HPLC or a similar technique. A suitable technique for determining the presence and amount of components (a) and (b) in the polymer film is provided in the Examples section below. Often during curing part of the solvent evaporates and the resulting film does not require any additional drying step.

Fluoro groups are not preferred because crosslinking agents having fluoro groups are less soluble in aqueous liquids and because of the environmental issue generally associated with fluorinated compounds. M⁺ is preferably an ammonium cation or an alkali metal cation, especially Li⁺. When M⁺ is Li⁺ the resultant components have particularly good solubility in water and aqueous liquids.

Component (a) is preferably of the Formula (Ia):

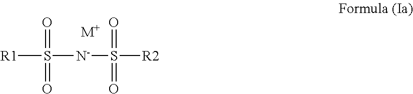

wherein at least one of R1 and R2, comprises one or more polymerisable groups, provided that the compound of Formula (Ia) comprises at least two polymerisable groups.

R1 and/or R2 may comprises non-polymerizable groups. Preferred non-polymerisable groups include amino, alkyl (especially C₁₋₄-alkyl) and aryl (especially phenyl or naphthyl), each of which is unsubstituted or carries one or more non-polymerisable substituents, e.g. C₁₋₄-alkyl, C₁₋₄-alkoxy, amino, C₁₋₄-alkyamine, sulpho, carboxy, or hydroxyl group.

Preferred polymerisable groups comprise ethylenically unsaturated groups, epoxide groups (e.g. glycidyl and epoxycyclohexyl groups), and thiol groups (e.g. alkylenethiol, preferably —C₁₋₃—SH). Optionally the polymerisable groups further comprise an optionally substituted alkylene group (e.g. optionally substituted C₁₋₆-alkylene) and/or an optionally substituted arylene group (e.g. optionally substituted C₆₋₁₈-arylene). The preferred substituents, when present, include C₁₋₄-alkyl, C₁₋₄-alkoxy, sulpho, carboxy, and hydroxyl groups.

Preferably component (a) is free from chloro groups.

Fluoro groups are F atoms covalently bound to a carbon atom. Likewise chloro groups are Cl atoms covalently bound to a carbon atom.

Preferred ethylenically unsaturated groups include vinyl groups, (meth)acrylic groups (e.g. CH₂═CR¹—C(O)— groups), especially (meth)acrylate groups (e.g. CH₂═CR¹—C(O)O— groups) and (meth)acrylamide groups (e.g. CH₂═CR¹—C(O)NR¹— groups), wherein each R¹ independently is H or CH₃). Most preferred ethylenically unsaturated groups comprise or are vinyl groups (CH₂═CH— groups).

Preferably component (a) is of Formula (II):

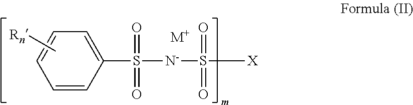

wherein:
n′ has a value of 1 or 2;
m has a value of 1 or 2;
M⁺ is a cation;
each R is independently a polymerisable or non-polymerisable group; and
X is an optionally substituted amine group, an optionally substituted alkylene group (e.g. optionally substituted C₁₋₆-alkylene) or an optionally substituted arylene group (e.g. optionally substituted C₆₋₁₈-arylene);
provided that the compound of Formula (II) comprises at least two polymerisable groups and is free from fluoro groups. For example, when m=1 and X is amino R is a polymerizable group and n′ is 2. Preferably the polymerizable groups comprise a vinyl group, epoxy group and/or thiol group. Thus R may be a vinyl group, epoxy group, thiol group or non-polymerisable group (as defined above).

The preferred substituents in X, when present, include C₁₋₄-alkyl, C₁₋₄-alkoxy, sulpho, carboxy, and hydroxyl groups.

In a preferred embodiment the compound of Formula (II) comprises 2, 3 or 4 polymerisable groups and especially 2 (and only 2) polymerisable groups.

In a preferred embodiment, component (a) is of Formula (II) wherein m and n′ both have a value of 1, X is a phenylene group carrying a vinyl group and R is a polymerizable group, preferably a vinyl group, and M⁺ is a cation.

In another preferred embodiment, component (a) is of Formula (II) wherein m has a value of 2, X is a C₁₋₆-alkylene, or C₆₋₁₈-arylene group or X is a group of the formula NR″ wherein each R″ independently is H or C₁₋₄-alkyl and R and M⁺ are as hereinbefore defined.

In a preferred embodiment, component (a) is of Formula (II) wherein m has a value of 1, n′ has a value of 2, X is a C₁₋₆-alkyl, or C₆₋₁₈-aryl group or a group of the formula N(R″)₂ wherein each R″ independently is H or C₁₋₄-alkyl and R is a polymerizable group, preferably a vinyl group, and M⁺ is a cation.

Illustrative synthesis methods for the above compounds of component (a) can be found below in the examples section below. Furthermore, many of the compounds of component (a) may be prepared by a process comprising the steps of:
(i) providing a sulfonyl halide (e.g. chloride, bromide or fluoride) compound;
(ii) reacting the sulfonyl halide group of component (i) with a compound comprising a sulfonamide group to obtain component (a) (e.g. of Formula (Ia) or (II));
wherein at least one of component (i) and component (ii) comprises at least one polymerisable group or a precursor thereof, preferably a vinyl group, epoxy group or thiol group. Preferably either component (i) or component (ii) comprises an aryl group, e.g. a phenylene group. For instance, component (i) may be a benzenesulfonyl chloride and component (ii) a sulfonamide,

In the illustrative synthesis method described above typically the vinyl group, epoxy group or thiol group is attached to a benzene ring of component (i) and/or (if present) of component (ii). In a preferred embodiment the sulfonyl halide compound used in the process comprises one or more vinyl groups, more preferably one or two vinyl groups, e.g. vinylbenzenesulfonyl halide or divinylbenzenesulfonyl halide.

As component (b) is non-ionic it cannot comprise a group of Formula (I).

The non-ionic crosslinking agent (b) is free from ionic groups, e.g. free from carboxylic acid, sulphonic acid and sulphonate groups.

In some embodiments the non-ionic crosslinking agent (b) comprises an aromatic group, e.g. a phenylene, naphthylene or triazine group. In other embodiments the non-ionic crosslinking agent (b) is free from aromatic groups.

In one embodiment the non-ionic crosslinking agent (b) is of Formula (III):

R′ₙ-A Formula (III)

wherein:
each R′ independently comprises a polymerisable group or a non-polymerisable group;
n has a value of 2, 3 or 4; and
A is a linking group;
provided that the compound of Formula (III) comprises at least two polymerisable groups and is free from ionic groups.

The polymerisable groups represented by R′ in Formula (Ill) include ethylenically unsaturated groups, epoxide groups (e.g. glycidyl and epoxycyclohexyl groups) and thiol groups (e.g. alkylenethiol, preferably —C₁₋₃—SH). Preferred ethylenically unsaturated groups are as described above in relation to R in Formula (II).

Component (b) may be obtained commercially or by methods known in the art.

Preferably the component (b) is free from fluoro and/or chloro groups.

A is preferably N (a nitrogen atom) or an optionally substituted alkylene group (e.g. optionally substituted C₁₋₆-alkylene) or an optionally substituted arylene group (e.g. optionally substituted C₆₋₁₈-arylene). The preferred substituents, when present, include C₁₋₄-alkyl, C₁₋₄-alkoxy and hydroxyl groups.

In a preferred embodiment, in Formula (III):
(i) n has a value of 2, 3 or 4 and A is C₁₋₆-alkylene or C₆₋₁₈-arylene; or
(ii) A is N, n has a value of 3 and either all three of the groups represented by R′ comprise a polymerisable group or two of the groups represented by R′ comprise a polymerisable group and the third group represented by R′ is H or C₁₋₄ alkyl; or
(iii) A is a triazine group or a cyanuric acid derivative, n has a value of 3 and either all three of the groups represented by R′ comprise a polymerisable group or two of the groups represented by R′ comprise a polymerisable group and the third group represented by R′ is C₁₋₄ alkoxy or C₁₋₄ alkyl.

As preferred examples of component (b) there may be mentioned compounds of the following formulae:

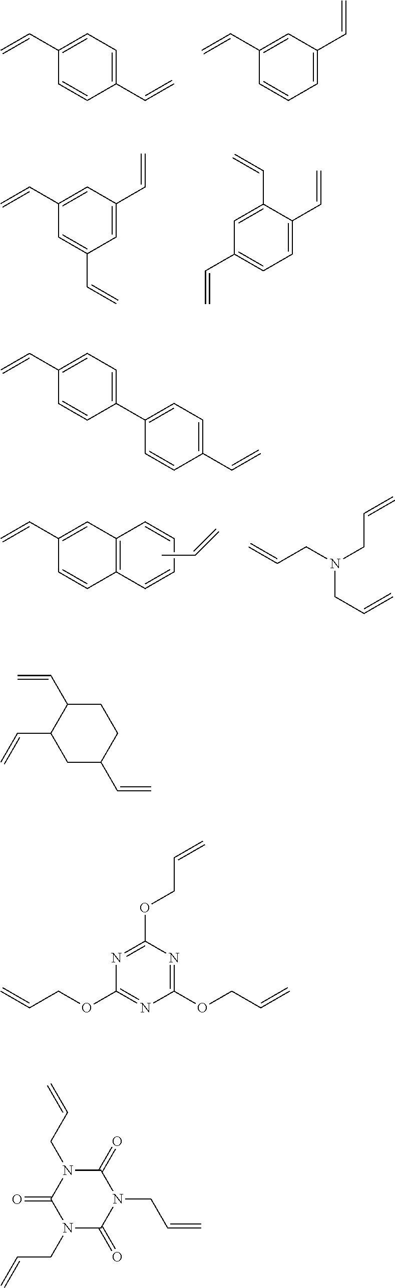

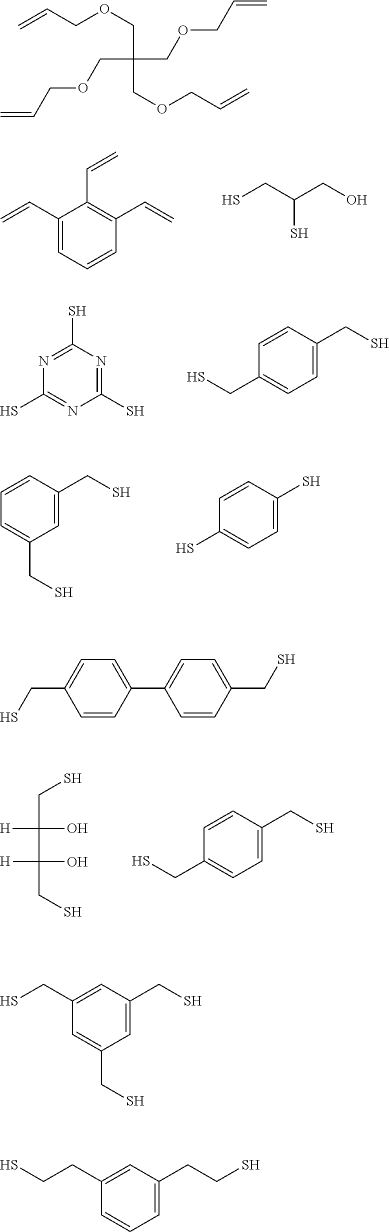

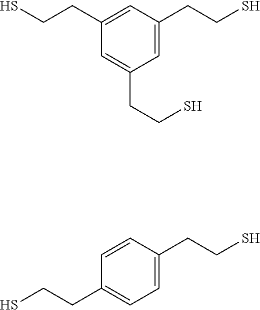 [SMILES: SCCc1cc(CCS)cc(CCS)c1 ; SCCc1ccc(CCS)cc1]

Preferably the polymerisable groups present in component (a) are copolymerisable with the polymerisable groups present in component (b). For example:
the polymerisable groups in one of components (a) and (b) are epoxy groups and the polymerisable groups in the other of components (a) and (b) are groups which are reactive with epoxy groups, e.g. thiol groups;
the polymerisable groups in components (a) and (b) are each independently selected from ethylenically unsaturated groups;
the polymerisable groups in one of components (a) and (b) are thiol groups and the polymerisable groups in the other of components (a) and (b) are groups which are reactive with thiol groups, e.g. ethylenically unsaturated groups.

Preferably component (b) comprises divinylbenzene, 2,4,6-triallyloxy-1,3,5-triazine, 1,3,5-triallylisocyanurate, triallylamine, 1,2,4-trivinylcyclohexane, tetra(allyloxy)ethane, pentaerythritol tetraallyl ether, 2,3-dimercapto-1-propanol, dithioerythritol, trithiocyanuric acid or a combination thereof.

The polymer film according to the first aspect of the present invention is preferably obtainable by curing a composition comprising:
(i) component (a) as defined above;
(ii) component (b) as defined above;
optionally (iii) a compound comprising one and only one polymerisable group;
optionally (iv) one or more radical initiators; and
optionally (v) a solvent.

The abovementioned composition forms a second aspect of the present invention.

Preferably, in some embodiments, the composition comprises 20 to 80 wt %, more preferably 30 to 70 wt %, of component (i) (i.e. component (a)).

Preferably the composition comprises 1 to 40 wt %, more preferably 2 to 20 wt %, most preferably 2 to 16 wt %, of component (ii) (i.e. component (b)).

Preferably component (ii) is liquid at 50° C. and has a boiling point over 90° C.

Preferably the composition comprises 0 to 40 wt %, more preferably 5 to 30% wt %, most preferably 6 to 25 wt %, of component (iii).

Preferably the composition comprises 0 to 10 wt %, more preferably 0.001 to 5 wt %, most preferably 0.005 to 2 wt %, of component (iv).

Preferably the composition comprises 0 to 40 wt %, more preferably 10 to 40 wt %, most preferably 15 to 30 wt %, of component (v).

The preferred polymerisable group which may be present in component (iii) is as defined above in relation to R, e.g. a vinyl group, e.g. in the form of allylic or styrenic group. Styrenic groups are preferred over e.g. (meth)acrylic groups as they increase the pH stability of the polymer film across the range of pH 0 to 14, which is of special interest when the polymer film is intended to be used as a cation exchange membrane for fuel cells.

Examples of compounds which may be used as component (iii) of the composition include the following compounds of Formula (MB-α), (AM-B) and Formula (IV):

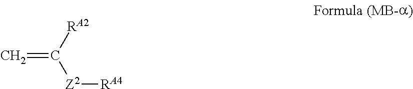

wherein in Formula (MB-α),
R^{A2} represents a hydrogen atom or an alkyl group,
R^{A4} represents an organic group comprising a sulfo group in free acid or salt form and having no ethylenically unsaturated group; and
Z² represents —NRa—, wherein Ra represents a hydrogen atom or an alkyl group preferably a hydrogen atom.

Examples of compounds of Formula (MB-α) include:

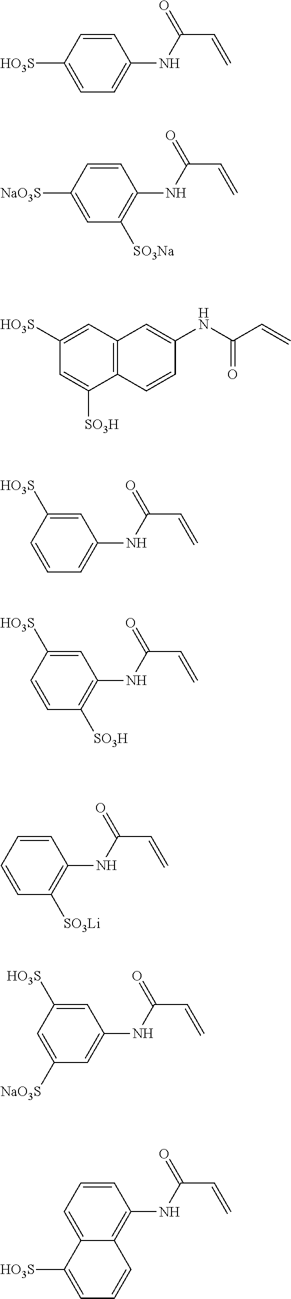

Synthesis methods for compounds of Formula (MB-α) can be found in e.g. US2015/0353696.

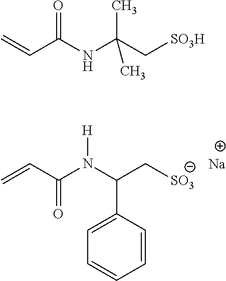

Synthesis methods for the above compounds can be found in e.g. US2016/0369017.

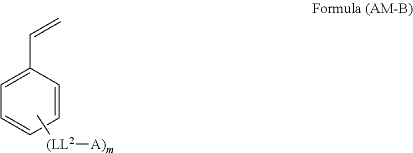

wherein in Formula (AM-B):
LL² represents a single bond or a bivalent linking group; and
A represents a sulfo group in free acid or salt form; and
m represents 1 or 2.
   Examples of compounds of Formula (AM-B) include:

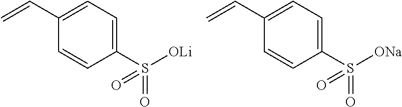

Such compounds of Formula (AM-B) are commercially available, e.g. from Tosoh Chemicals and Sigma-Aldrich.

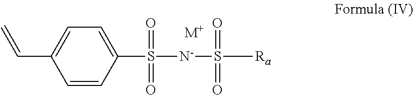

wherein
Rₐ in Formula (IV) is C₁₋₄ alkyl, NH₂, C₆₋₁₂-aryl; and
M⁺ is a cation, preferably H⁺, Li⁺, Na⁺, K⁺, NL₄⁺ wherein each L independently is H or C₁₋₃-alkyl.

Examples of compounds of Formula (IV) include:

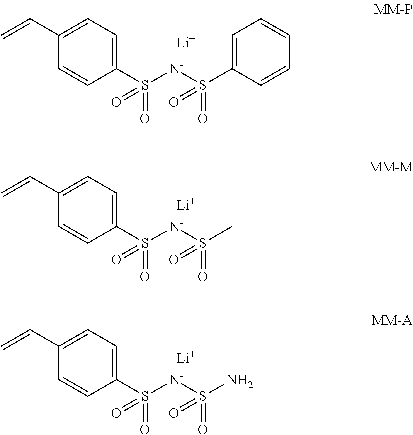

Synthesis methods for the above compounds having the MM prefix are described in the Examples section below.

Preferably component (iii) is chosen from the compounds of Formula (AM-B) and/or Formula (IV) because this can result in polymer films having especially good stability in the pH range 0 to 14.

The radical initiator is preferably a thermal initiator or a photoinitiator.

Examples of suitable thermal initiators which may be used as component (iv) include 2,2′-azobis(2-methylpropionitrile) (AIBN), 4,4′-azobis(4-cyanovaleric acid), 2,2′-azobis(2,4-dimethyl valeronitrile), 2,2′-azobis(2-methylbutyronitrile), 1,1′-azobis(cyclohexane-1-carbonitrile), 2,2′-azobis(4-methoxy-2,4-dimethyl valeronitrile), dimethyl 2,2′-azobis(2-methylpropionate), 2,2′-azobis[N-(2-propenyl)-2-methylpropionamide, 1-[(1-cyano-1-methylethyl)azo]formamide, 2,2′-Azobis(N-butyl-2-methylpropionamide), 2,2′-Azobis(N-cyclohexyl-2-methylpropionamide), 2,2′-Azobis(2-methylpropionamidine) dihydrochloride, 2,2′-Azobis[2-(2-imidazolin-2-yl)propane]dihydrochloride, 2,2′-Azobis[2-(2-imidazolin-2-yl)propane]disulfate dihydrate, 2,2′-Azobis[N-(2-carboxyethyl)-2-methylpropionamidine] hydrate, 2,2′-Azobis{2-[1-(2-hydroxyethyl)-2-imidazolin-2-yl]propane} dihydrochloride, 2,2′-Azobis[2-(2-imidazolin-2-yl)propane], 2,2′-Azobis(1-imino-1-pyrrolidino-2-ethylpropane) dihydrochloride, 2,2′-Azobis{2-methyl-N-[1,1-bis(hydroxymethyl)-2-hydroxyethl]propionamide} and 2,2′-Azobis[2-methyl-N-(2-hydroxyethyl)propionamide].

Examples of suitable photoinitiators which may be included in the composition as component (iv) include aromatic ketones, acylphosphine compounds, aromatic onium salt compounds, organic peroxides, thio compounds, hexa-arylbiimidazole compounds, ketoxime ester compounds, borate compounds, azinium compounds, metallocene compounds, active ester compounds, compounds having a carbon halogen bond, and an alkyl amine compounds. Preferred examples of the aromatic ketones, the acylphosphine oxide compound, and the thio-compound include compounds having a benzophenone skeleton or a thioxanthone skeleton described in “RADIATION CURING IN POLYMER SCIENCE AND TECHNOLOGY”, pp. 77-117 (1993). More preferred examples thereof include an alpha-thiobenzophenone compound described in JP1972-6416B (JP-S47-6416B), a benzoin ether compound described in JP1972-3981B (JP-S47-3981B), an alpha-substituted benzoin compound described in JP1972-22326B (JP-S47-22326B), a benzoin derivative described in JP1972-23664B (JP-S47-23664B), an aroylphosphonic acid ester described in JP1982-30704A (JP-S57-30704A), dialkoxybenzophenone described in JP1985-26483B (JP-S60-26483B), benzoin ethers described in JP1985-26403B (JP-S60-26403B) and JP1987-81345A (JPS62-81345A), alpha-amino benzophenones described in JP1989-34242B (JP H01-34242B), U.S. Pat. No. 4,318,791A, and EP0284561A1, p-di(dimethylaminobenzoyl) benzene described in JP1990-211452A (JP-H02-211452A), a thio substituted aromatic ketone described in JP1986-194062A (JPS61-194062A), an acylphosphine sulfide described in JP1990-9597B (JP-H02-9597B), an acylphosphine described in JP1990-9596B (JP-H02-9596B), thioxanthones described in JP1988-61950B (JP-S63-61950B), and coumarins described in JP1984-42864B (JP-S59-42864B). In addition, the photoinitiators described in JP2008-105379A and JP2009-114290A are also preferable. In addition, photoinitiators described in pp. 65 to 148 of “Ultraviolet Curing System” written by Kato Kiyomi (published by Research Center Co., Ltd., 1989) may be used.

Especially preferred photoinitiators include Norrish Type II photoinitiators having an absorption maximum at a wavelength longer than 380 nm, when measured in one or more of the following solvents at a temperature of 23° C.: water, ethanol and toluene. Examples include a xanthene, flavin, curcumin, porphyrin, anthraquinone, phenoxazine, camphorquinone, phenazine, acridine, phenothiazine, xanthone, thioxanthone, thioxanthene, acridone, flavone, coumarin, fluorenone, quinoline, quinolone, naphtaquinone, quinolinone, arylmethane, azo, benzophenone, carotenoid, cyanine, phtalocyanine, dipyrrin, squarine, stilbene, styryl, triazine or anthocyanin-derived photoinitiator.

Preferably component (v) of the composition is an inert solvent. In other words, preferably component (v) does not react with any of the other components of the composition. In one embodiment the component (v) preferably comprises water and optionally an organic solvent, especially where some or all of the organic solvent is water-miscible. The water is useful for dissolving component (i) and possibly also component (iii) and the organic solvent is useful for dissolving component (ii) or any other organic components present in the composition.

Component (v) is useful for reducing the viscosity and/or surface tension of the composition. In some embodiments, the composition comprises 10 to 40 wt %, especially 15 to 30 wt %, of component (v).

Examples of inert solvents which may be used as or in component (v) include water, alcohol-based solvents, ether based solvents, amide-based solvents, ketone-based solvents, sulphoxide-based solvents, sulphone-based solvents, nitrile-based solvents and organic phosphorus based solvents. Examples of alcohol-based solvents which may be used as or in component (v) (especially in combination with water) include methanol, ethanol, isopropanol, n-butanol, ethylene glycol, propylene glycol, diethylene glycol, dipropylene glycol and mixtures comprising two or more thereof. In addition, preferred inert, organic solvents which may be used in component (v) include dimethyl sulphoxide, dimethyl imidazolidinone, sulpholane, N-methylpyrrolidone, dimethyl formamide, acetonitrile, acetone, 1,4-dioxane, 1,3-dioxolane, tetramethyl urea, hexamethyl phosphoramide, hexamethyl phosphorotriamide, pyridine, propionitrile, butanone, cyclohexanone, tetrahydrofuran, tetrahydropyran, 2-methyltetrahydrofuran, ethylene glycol diacetate, cyclopentylmethylether, methylethylketone, ethyl acetate, y-butyrolactone and mixtures comprising two or more thereof. Dimethyl sulphoxide, N-methyl pyrrolidone, dimethyl formamide, dimethyl imidazolidinone, sulpholane, acetone, cyclopentylmethylether, methylethylketone, acetonitrile, tetrahydrofuran, 2-methyltetrahydrofuran and mixtures comprising two or more thereof are preferable.

The solvents mentioned above may also be used to extract components (a) and (b) from the polymer film for determining their content.

Preferably components (a) and (b) (i.e. (i) and (ii) of the composition) can polymerize by radiation, thermal or electron beam initiation. When component (a) or (b) comprises an ethylenically unsaturated group, epoxy group, or thiol group, especially an ethylenically unsaturated group (e.g. a vinyl group), such group is preferably attached to an aromatic carbon atom such as a benzene ring, e.g. as in divinylbenzene. When component (a) or (b) comprises a thiol group such group is preferably attached to a non-aromatic carbon atom.

Preferably the composition according to the second aspect of the present invention comprises:
1) 20 to 80 wt % of component (i);
2) 1 to 40 wt % of component (ii);
3) 0 to 40 wt % of component (iii);
4) 0 to 10 wt % of component (iv); and
5) 0 to 40 wt % of component (v).

According to a third aspect of the present invention there is provided a process for preparing a polymer film comprising curing of a composition according to the second aspect of the present invention.

The process for preparing the polymer film preferably comprises the steps of:
i. providing a porous support;
ii. impregnating the porous support with the composition of the second aspect of the present invention; and
iii. curing the curable composition.

The preferences for the composition used in the process of the third aspect of the present invention are as described herein in relation to the second aspect of the present invention.

Preferably the polymer film arising from the process according to the third aspect of the present invention comprises 0.008 mg/g to 25 mg/g of component (a), more preferably 0.015 to 20 mg/g of component (a).

Preferably the polymer film arising from the process according to the third aspect of the present invention comprises 0.008 to 25 mg/g, more preferably 0.015 to 20 mg/g of component (b).

The compositions may be cured by any suitable process, including thermal curing, photocuring, electron beam (EB) irradiation, gamma irradiation, and combinations of the foregoing.

Preferably the process according to the third aspect of the present invention comprises a first curing step and a second curing step (dual curing). In a preferred embodiment the compositions are cured first by photocuring, e.g. by irradiating the compositions by ultraviolet or visible light, or by gamma or electron beam radiation, and thereby causing the curable components present in the compositions to polymerise, and then applying a second curing step. The second curing step preferably comprises thermal curing, gamma irradiation or EB irradiation whereby the second curing step preferably applies a different method than the first curing step. When gamma or electron beam irradiation is used in the first curing step preferably a dose of 60 to 120 kGy, more preferably a dose of 80 to 100 kGy.

In one embodiment the process according to the third aspect of the present invention comprises curing the composition in a first curing step (e.g. UV curing or electron beam (EB) curing) to form a polymer film, winding the polymer film onto a core (optionally together with an inert polymer foil) and then performing a second curing step (e.g. thermal curing). In another embodiment the process comprises curing the composition in a first curing step (e.g. UV curing) to form a polymer film, performing a second curing step (e.g. EB curing) and then winding the polymer film onto a core (optionally together with an inert polymer foil).

In a preferred embodiment the first and second curing steps are respectively selected from (i) UV curing then thermal curing; (ii) UV curing then electron beam curing; and (iii) electron beam curing then thermal curing.

The composition preferably comprises 0.05 to 5 wt % of component (iv) for the first curing step. The composition optionally further comprises 0 to 5 wt % of a second component (iv) for the second curing step. When it is intended to cure the composition thermally or using light (e.g. UV or visible light) the composition preferably comprises 0.001 to 2 wt %, depending on the selected radical initiator, in some embodiments 0.005 to 0.9 wt %, of component (iv). Component (iv) may comprise more than one radical initiator, e.g. a mixture of several photoinitiators (for single curing) or a mixture of photoinitiators and thermal initiators (for dual curing). Alternatively a second curing step is performed using gamma or EB irradiation. For the second curing step by gamma or EB irradiation preferably a dose of 20 to 100 kGy is applied, more preferably a dose of 40 to 80 kGy.

For the second curing step, thermal curing is preferred. The thermal curing is preferably performed at a temperature between 50 and 100° C., more preferably between 60 and 90° C. The thermal curing is preferably performed for a period between 2 and 48 hours, e.g. between 8 and 16 hours, e.g. about 10 hours. Optionally after the first curing step a polymer foil is applied to the polymer film before winding (this reduces oxygen inhibition and/or sticking of the polymer film onto itself).

Preferably the process according to the third aspect of the present invention is performed in the presence of a porous support. For example, the composition according to the second aspect of the present invention is present in and/or on a porous support. The porous support provides mechanical strength to the polymer film resulting from curing the composition according to the second aspect of the present invention and this is particularly useful when the polymer film is intended for use as a CEM or BPM.

As examples of porous supports which may be used there may be mentioned woven and non-woven synthetic fabrics and extruded films. Examples include wetlaid and drylaid non-woven material, spunbond and meltblown fabrics and nanofiber webs made from, e.g. polyethylene, polypropylene, polyacrylonitrile, polyvinyl chloride, polyphenylenesulfide, polyester, polyamide, polyaryletherketones such as polyether ether ketone and copolymers thereof. Porous supports may also be porous membranes, e.g. polysulphone, polyethersulphone, polyphenylenesulphone, polyphenylenesulfide, polyimide, polyethermide, polyamide, polyamideimide, polyacrylonitrile, polycarbonate, polyacrylate, cellulose acetate, polypropylene, poly(4-methyl 1-pentene), polyinylidene fluoride, polytetrafluoroethylene, polyhexafluoropropylene and polychlorotrifluoroethylene membranes and derivatives thereof.

The porous support preferably has an average thickness of between 10 and 800 μm, more preferably between 15 and 300 μm, especially between 20 and 150 μm, more especially between 30 and 130 μm, e.g. around 60 μm or around 100 μm.

Preferably the porous support has a porosity of 30 and 95%. The porosity of the support may be determined by a porometer, e.g. a Porolux™ 1000 from 113-FT GmbH, Germany.

The porous support, when present, may be treated to modify its surface energy, e.g. to values above 45 mN/m, preferably above 55 mN/m. Suitable treatments include corona discharge treatment, plasma glow discharge treatment, flame treatment, ultraviolet light irradiation treatment, chemical treatment or the like, e.g. for the purpose of improving the wettability of and the adhesiveness to the porous support to the polymer film.

Commercially available porous supports are available from a number of sources, e.g. from Freudenberg Filtration Technologies (Novatexx materials), Lydall Performance Materials, Celgard LLC, APorous Inc., SWM (Conwed Plastics, DelStar Technologies), Teijin, Hirose, Mitsubishi Paper Mills Ltd and Sefar AG.

Preferably the porous support is a porous polymeric support. Preferably the porous support is a woven or non-woven synthetic fabric or an extruded film without covalently bound ionic groups.

In a preferred process according to the third aspect of the present invention, the composition according to the second aspect of the present invention may be applied continuously to a moving (porous) support, preferably by means of a manufacturing unit comprising a composition application station, one or more irradiation source(s) for curing the composition, a polymer film collecting station and a means for moving the support from the composition application station to the irradiation source(s) and to the polymer film collecting station.

The composition application station may be located at an upstream position relative to the irradiation source(s) and the irradiation source(s) is/are located at an upstream position relative to the polymer film collecting station.

Examples of suitable coating techniques for applying the composition according to the second aspect of the present invention to a porous support include slot die coating, slide coating, air knife coating, roller coating, screen-printing, and dipping. Depending on the used technique and the desired end specifications, it might be desirable to remove excess coating from the substrate by, for example, roll-to-roll squeeze, roll-to-blade or blade-to-roll squeeze, blade-to-blade squeeze or removal using coating bars. Curing by light is preferably done for the first curing step, preferably at a wavelength between 300 nm and 800 nm using a dose between 40 and 20000 mJ/cm². In some cases additional drying might be needed for which temperatures between 40° C. and 200° C. could be employed. When gamma or EB curing is used irradiation may take place under low oxygen conditions, e.g. below 200 ppm oxygen.

According to a fourth aspect of the present invention there is provided a polymer film obtainable or obtained by the process of the third aspect of the present invention.

Preferably the polymer film is a cation exchange membrane (CEM) obtained from polymerising the composition according to the second aspect of the present invention, and/or by a process according to the third aspect of the present invention.

The polymer film according the present invention preferably has a very high density. A high density may be achieved by preparing the polymer film from the composition according to the second aspect of the present invention having a low amount of component (v). Thus the present invention enables the production of high density polymer films (e.g. CEMs) having a very high ion exchange capacity and therefore low electrical resistance.

According to a fifth aspect of the present invention there is provided use of the polymer film (e.g. a cation exchange membrane) according to the first or fourth aspect of the present invention for the treatment of a polar liquid (e.g. to remove salts or to purify a polar liquid) or for the generation of electricity (e.g. by reverse electrodialysis).

### EXAMPLES

In the following non-limiting examples all parts and percentages are by weight unless specified otherwise.

TABLE 1
Ingredients used in the Examples:

| Abbreviation | Component Type | Description/supplier |
|---|---|---|
| XL-B | (a) and (i) | Benzenesulfonamide, 4-ethenyl-N-[(4- |
| | | ethenylphenyl)sulfonyl]-, lithium salt |
| XL-D | (a) and (i) | Benzenesulfonamide, 2,4-diethenyl-N-(methylsulfonyl)-, |
| | | lithium salt |
| XL-E | (a) and (i) | Benzenesulfonamide, 2,4-diethenyl-N-[(2,4- |
| | | diethenylphenyl)sulfonyl]-, lithium salt |
| XL-P | (a) and (i) | Benzenesulfonamide, 2,4-diethenyl-N-(phenylsulfonyl)-, |
| | | lithium salt |
| XL-2 | (a) and (i) | 1,3-[N-(ethenylphenylsulfonyl)benzene sulfonamide], |
| | | dilithium salt |
| XL-3 | (a) and (i) | Benzenesulfonamide, 4-(2-mercaptoethyl)-N-[[4-(2- |
| | | hydroxyethyl)phenyl]sulfonyl]-, lithium salt |
| DVB | (b) and (ii) | Divinylbenzene from Sigma-Aldrich |
| TAOT | (b) and (ii) | 2,4,6-triallyloxy-1,3,5-triazine from Sigma-Aldrich |
| PETAE | (b) and (ii) | pentaerythritol tetraallyl ether from Fujifilm-Wako |
| | | Chemicals |
| TVCH | (b) and (ii) | 1,2,4-trivinylcyclohexane from Sigma-Aldrich |
| TCA | (b) and (ii) | Trithiocyanuric acid |
| MM-M | (iii) | Benzenesulfonamide, 4-ethenyl-N-(methylsulfonyl)-, |
| | | lithium salt |
| MM-P | (iii) | Benzenesulfonamide, 4-ethenyl-N-(phenylsulfonyl)-, |
| | | lithium salt |
| LAP | (iv) | phenyl-2,4,6-trimethylbenzoylphosphinate, lithium salt |
| | | from Sigma-Aldrich |
| TPO-L | (iv) | (ethyl(2,4,6-trimethylbenzoyl)-phenyl phosphinate from |
| | | Omnirad |
| Irgacure ™ | (iv) | 2-Hydroxy-2-methyl-1-phenyl-propan-1-one from BASF |
| 1173 | | |
| DBzO | (iv) | Dibenzoyl peroxide from Sigma-Aldrich |
| V-601 | (iv) | Dimethyl 2,2′-azobis(2-methylpropionate) from Fujifilm- |
| | | Wako Chemicals |
| V-40 | (iv) | 1,1′-Azobis(cyclohexane-1-carbonitrile) from Fujifilm- |
| | | Wako Chemicals |
| AC-044 | (iv) | 2,2′-Azobis[2-(2-imidazolin-2-yl)propane] dihydrochloride |
| | | from Fujifilm-Wako Chemicals |
| MeOH | (v) | Methanol, from Sigma-Aldrich |
| IPA | (v) | Isopropanol from Sigma-Aldrich |
| MCH | (v) | Methylcyclohexane from Sigma-Aldrich |
| NMP | (v) | N-methylpyrrole from Sigma-Aldrich |
| DMSO | (v) | Dimethylsulfoxide from Sigma-Aldrich |
| 4OH-TEMPO | | 4-hydroxy-2,2,6,6-tetramethylpiperidin-1-oxyl, from |
| | | Sigma-Aldrich |
| LiSS | | Styrene sulfonate, lithium salt from Tosoh chemicals |
| DVBS-Na | | Divinylbenzene sulfonate, sodium salt from Tosoh |
| | | chemicals |
| Cl-SS | | Styrene sulfonate, chloride |
| Cl-DVBS | | Divinylbenzene sulfonate, chloride |
| NH2-SS | | Styrene sulfonamide |
| THF | | Tetrahydrofuran from Sigma-Aldrich |
| DMF | | Dimethylformamide from Sigma-Aldrich |
| DCM | | Dichloromethane from Sigma-Aldrich |
| KCl | | Potassium chloride from Sigma-Aldrich |
| LiH | | Lithium hydride from Sigma-Aldrich |
| Celite | | Celite S, diatomaceous earth (SiO₂) from Sigma-Aldrich |

XL-B, XL-D, XL-E, XL-P, XL-2 and XL-3 had the structures shown below:

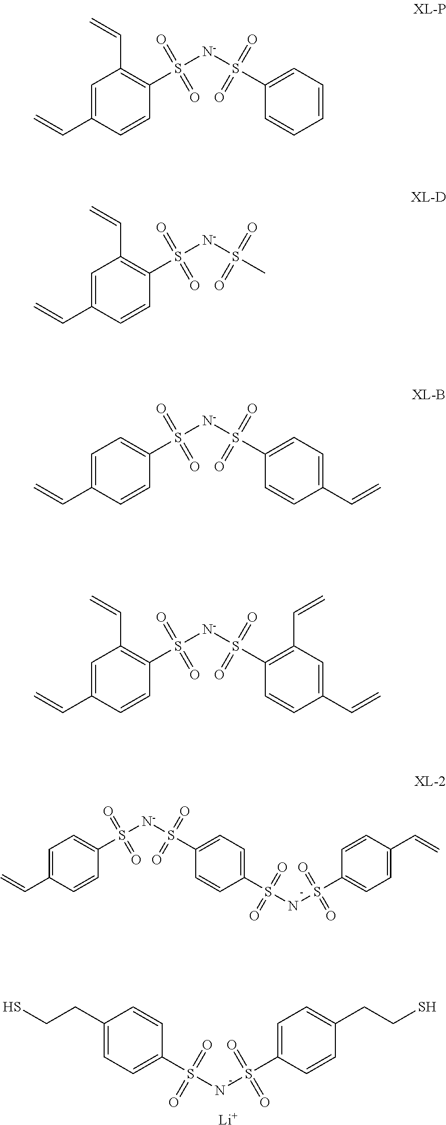

### XL-3

### Methods

### Determination of the Amount of Component (a) and (b) in the Polymer Film

This method determines the amount of component (a) and (b) present in the polymer films under test by HPLC analysis of ethanol extracts from the polymer films. The analysis was performed as follows: a 200 mg sample of the polymer film under test (taken from an aluminum bag) was put in a Falcon® tube and 15 ml of ethanol was added. The tube was closed tightly and shaken overnight (about 16 hr), at room temperature (20° C.). The solution was then filtered using a 25 mm HPLC Syringe filter, 0.45 μm RC (from BGB Analytik) and the quantity of (unpolymerised) components (a) and (b) present in the ethanol was measured by HPLC. The HPLC analysis used 5 point calibration lines made from 10 to 500 mg/l for each of components (a) and (b) used to make the polymer film under test.

The HPLC apparatus used was a Waters Acquity UPLC equipped with a PDA detector at 254 nm fitted with a Waters Xbridge C8 150 mm 4.6 mm column. The components (a) and (b) were eluted using a mixture of water and methanol, each containing a 0.1 wt % of acetic acid. The gradient used is described in Table 2 in which A is water and B is methanol. The results, expressed in mg of extracted amount of compound (a) or compound (b) per gram of polymer film, are given in Table 4.

TABLE 2
HPLC Gradient

| Time | Flow | A | B | |
|---|---|---|---|---|
| [min] | [ml/min] | [%] | [%] | Curve |
| Initial | 0.5 | 95.0 | 5.0 | Initial |
| 1.20 | 0.5 | 95.0 | 5.0 | 6 |
| 30.00 | 0.5 | 21.5 | 78.5 | 6 |
| 36.00 | 0.5 | 0.0 | 100.0 | 6 |
| 39.60 | 0.5 | 0.0 | 100.0 | 6 |
| 39.72 | 0.5 | 95.0 | 5.0 | 6 |
| 43.20 | 0.5 | 95.0 | 5.0 | 6 |

### Measurement of Electrical Resistance (ER)

ER (ohm·cm²) of the polymer films prepared in the Examples was measured by the method described by Dlugolecki et al., J. of Membrane Science, 319 (2008) on page 217-218 with the following modifications:
the auxiliary polymer films were CMX and AMX from Tokuyama Soda, Japan;
the capillaries as well as the Ag/AgCl references electrodes (Metrohm type 6.0750.100) contained 3M KCl;
the calibration liquid and the liquid in compartment 2, 3, 4 and 5 was 0.5 M NaCl solution at 25° C.;
the effective polymer film area was 9.62 cm²;
the distance between the capillaries was 5.0 mm;
the measuring temperature was 25° C.;
a Cole Parmer Masterflex console drive (77521-47) with easy load II model 77200-62 gear pumps was used for all compartments;
the flowrate of each stream was 475 ml/min controlled by Porter Instrument flowmeters (type 150AV-B250-4RVS) and Cole Parmer flowmeters (type G-30217-90); and
the samples were equilibrated for at least 1 hour at room temperature in a 0.5 M solution of NaCl prior to measurement.

### Measurement of Permselectivity (PS)

The permselectivity PS (%) that is the selectivity to the passage of ions of opposite charge to that of the polymer films prepared in the examples, was measured as follows. The polymer film to be analyzed was placed in a two-compartment system. One compartment is filled with a 0.05M solution of NaOH and the other with a 0.5M solution of NaOH.

### Settings

the capillaries as well as the Ag/AgCl reference electrodes (Metrohm type 6.0750.100) contained 3M KCl;
the effective polymer film area was 9.62 cm²;
the distance between the capillaries was ca 15 mm;
the measuring temperature was 21.0±0.2° C.;
a Cole Parmer Masterflex console drive (77521-47) with easy load II model 77200-62 gear pumps was used for the two compartments;
Porter Instrument flowmeters (type 150AV-B250-4RVS) and Cole Parmer flowmeters (type G-30217-90) were used to control the flow constant at 500 ml/min;
the samples were equilibrated for 1 hr in a 0.5M NaOH solution prior to measurement. The voltage was read from a regular VOM (multitester) after 20 minutes.

Preferably the PS for NaOH is at least 85%.

### Measurement of Polymer Film Swelling

The swelling of the polymer films was measured as follows:

A piece of the polymer film under test was immersed in water for 24 hrs. After that period the excess water was wiped-off using a paper towel and the polymer film was weighed. The wet polymer film was then placed in an oven at 40° C. for 15 hr until it was totally dry. Then the polymer film was weighed again and the swelling is calculated as follows:

$$Swelling = \frac{{M}_{w e t - {M}_{dry}}}{{M}_{dry}} \times 100$$

wherein M_{wet} and M_{dry} are the masses of the polymer film wet and dry, respectively.

### Preparation of Component (a)

### Synthesis of Monomers and Starting Materials

### Cl-SS

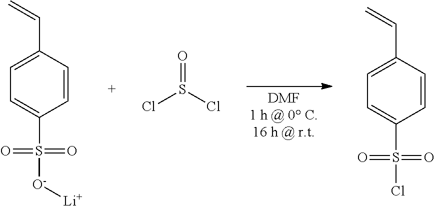

Thionyl chloride (109 mL, 178.46 g, 1.5 mol, 3 moleq) was added dropwise to a solution of 4-vinylbenzenesulfonic acid lithium salt (95.08 g, 0.500 mol, 1 moleq) and 4OH-TEMPO (50 mg, 500 ppm) in DMF (300 mL) in a double-walled reactor that was actively cooled to 5° C. After the addition was completed, the solution was allowed to slowly heat to room temperature and was stirred for another 16 hours. Then the reaction mixture was poured into 1 litre of cold 1M KCl in a separation funnel. The bottom layer was removed and dissolved in 500 mL diethylether. This solution was washed with a 1M KCl-solution (300 mL). The organic layer was dried over sodium sulfate, filtered and concentrated in vacuum to give a yellow oil. The crude product was used without further purification in the next step. Typical yield is 89.5 g (88%). HPLC-MS purity>98%; ¹H-NMR: <2 wt % DMF, 0% diethyl ether.

### Cl-DVBS

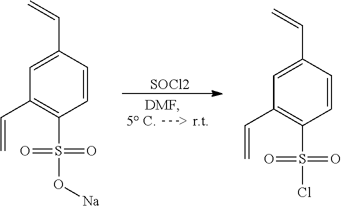

Thionyl chloride (75 mL, 123.1 g, 1.034 mol, 3 moleq) was added dropwise to an solution of divinylbenzene sulphonate sodium salt (80 g, 0.345 mol, 1 moleq) and 4OH-TEMPO (50 mg, 500 ppm) in DMF (300 mL) in a double-walled reactor that was actively cooled to 5° C. After the addition was completed, the solution was allowed to slowly heat to room temperature and was stirred for another 16 hours. Then the reaction mixture was poured into 1 liter of cold 1M KCl in a separation funnel. The bottom layer was removed and dissolved in 500 mL diethylether. This solution was washed with a 1M KCl-solution (300 mL). The organic layer was dried over sodium sulfate, filtered and concentrated in vacuo to give a yellow oil. The crude product was used without further purification in the next step. Typical yield is 62 g (79%). HPLC-MS purity>98%; ¹H-NMR: <2 wt % DMF, 0% diethyl ether.

### NH2-SS

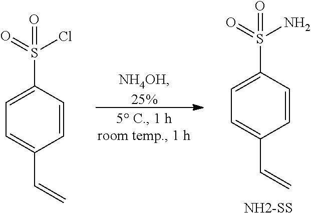

Thionyl chloride (109 mL, 178.46 g, 1.5 mol, 3 moleq) was added dropwise to a solution of 4-vinylbenzene-sulfonic acid lithium salt (95.08 g, 0.500 mol, 1 moleq) and 4OH-TEMPO (50 mg, 500 ppm) in DMF (300 mL) in a double-walled reactor that was actively cooled to 5° C. After the addition was completed, the solution was allowed to slowly heat to room temperature and was stirred for another 16 hours. Then the reaction mixture was poured into 1 litre of cold 1M KCl in a separation funnel. The bottom layer was removed and was added dropwise to a solution of ammonium hydroxide 25% in water (250 mL, 3.67 mol, 15 moleq) and 4OH-TEMPO (50 mg, 500 ppm) in a double-walled reactor that was actively cooled to 5° C. After the addition was completed, the 10 solution was stirred for 1 hour. The solution was then allowed to heat to room temperature and was stirred for one hour. Then the reaction mixture was cooled back to 5° C. and the product was filtered off and washed with 50 mL of cold water. The product was dried overnight in vacuum at 30° C. and used without further purification. Typical yield was 66.8 g (73%). HPLC-MS purity>95%.

### NH2-DVBS

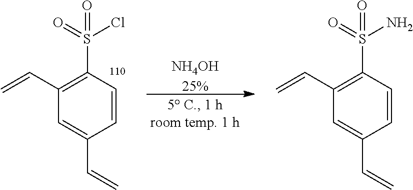

Cl-DVBS was added dropwise to a solution of ammonium hydroxide 25% in water (250 mL, 3.67 mol, 15 moleq) and 4OH-TEMPO (50 mg, 500 ppm) in a double-walled reactor that was actively cooled to 5° C. After the addition was completed, the solution was stirred for 1 hour. The solution was then allowed to heat to room temperature and was stirred for one hour. Then the reaction mixture was cooled back to 5° C. and the product was filtered off and washed with 50 mL of cold water. The product was dried overnight in vacuum at 30° C. and used without further purification. Typical yield was 66.8 g (70%). HPLC-MS purity>95%.

### Synthesis of Compounds of Component (a)/(i):

### XL-D

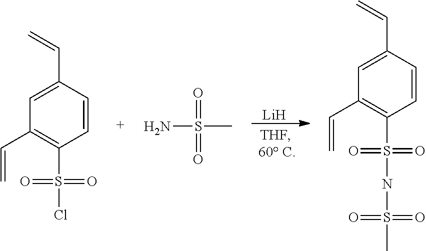

Before the synthesis, methane sulfonamide was dried in a vacuum oven overnight (30° C., vac). To a solution of the dried methane sulfonamide (8.32 g, 0.087 mol, 1 moleq) and 4OH-TEMPO (30 mg, 500 ppm) in THF (100 mL) was added LiH (1.53 g, 0.192 mol, 2.2 moleq) as a solid at once. The reaction mixture was stirred for 30 minutes at room temperature. Then, a solution of Cl-DVBS (20 g, 0.087 mol, 1 moleq) in THF (50 mL) was added to the reaction mixture. After addition, the reaction mixture was heated to 60° C. (water bath temperature). After two days, the reaction mixture was filtrated over celite to remove the excess of LiH. The filtrate was concentrated in vacuo to give a light-yellow foam. The resulting foam was dissolved in 500 mL ethyl acetate. Celite was added and the resulting slurry was stirred for 5 minutes. Then, the celite was filtered off and washed with 100 mL ethyl acetate. This Celite procedure was then repeated. The solvent was then evaporated in vacuo and the resulting white foam was washed with 500 mL diethyl ether overnight. The resulting white powder was filtered off and dried in a vacuum oven at 30° C. for 16 h yielding a hygroscopic white solid. Typical achieved yield is 15.5 g (60%). HPLC-MS purity>95%; ¹H-NMR: <3 wt % residual solvents; 2 wt % divinylbenzene sulphonate; ICP-OES: 24-30 g Li/kg product.

### XL-P

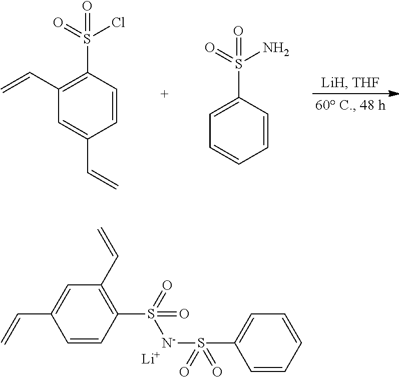

Before the synthesis, benzene sulfonamide was dried in a vacuum oven overnight (30° C., vac). To a solution of the dried benzene sulfonamide (0.087 mol, 1 moleq) and 40H-TEMPO (30 mg, 500 ppm) in THF (100 mL) was added LiH (0.192 mol, 2.2 moleq) as a solid at once. The reaction mixture was stirred for 30 minutes at room temperature. Then, a solution of Cl-DVBS (0.087 mol, 1 moleq) in THF (50 mL) was added to the reaction mixture. After addition, the reaction mixture was heated to 60° C. (water bath temperature). After two days, the reaction mixture was filtrated over celite to remove the excess of LiH. The filtrate was concentrated in vacuo to give a light-yellow foam. The resulting foam was dissolved in 500 mL ethyl acetate. Celite was added and the resulting slurry was stirred for 5 minutes. Then, the celite was filtered off and washed with 100 mL ethyl acetate. This Celite procedure was then repeated. The solvent was then evaporated in vacuo and the resulting white foam was washed with 500 mL diethyl ether overnight. The resulting white powder was filtered off and dried in a vacuum oven at 30° C. for 16 h yielding a hygroscopic white solid. Typical achieved yield is 60%. HPLC-MS purity>95%; ¹H-NMR: <3 wt % residual solvents; 2 wt % divinylbenzene sulphonate; ICP-OES: 24-30 g Li/kg product.

### XL-E

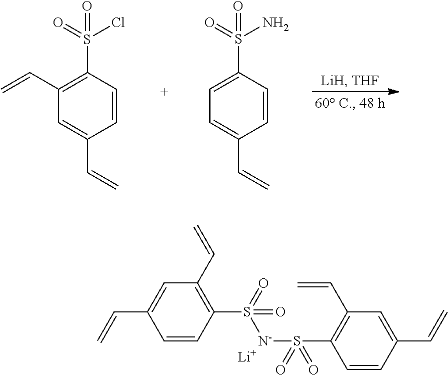

Before the synthesis, benzene sulphonamide was dried in a vacuum oven overnight (30° C., vac). To a solution of the dried benzene sulfonamide (0.061 mol, 1 moleq) and 4OH-TEMPO (30 mg, 500 ppm) in THF (100 mL) was added LiH (0.134 mol, 2.2 moleq) as a solid at once. The reaction mixture was stirred for 30 minutes at room temperature. Then, a solution of Cl-DVBS (0.061 mol, 1 moleq) in THF (50 mL) was added to the reaction mixture. After addition, the reaction mixture was heated to 60° C. (water bath temperature). After two days, the reaction mixture was filtrated over celite to remove the excess of LiH. Celite was added and the resulting slurry was stirred for 5 minutes. Then, the celite was filtered off and washed with 100 mL ethyl acetate. The solvent was then evaporated in vacuo and the resulting white foam was washed with 500 mL diethyl ether overnight. The resulting white powder was filtered off and dried in a vacuum oven at 30° C. for 16 h yielding a white solid. Typical yield is 51%. HPLC-MS purity>94%; ¹H-NMR: <1 wt % residual solvents, <5 wt % styrene sulphonate or styrene sulphonamide; ICP-OES: 21-26 g Li/kg product.

### XL-B

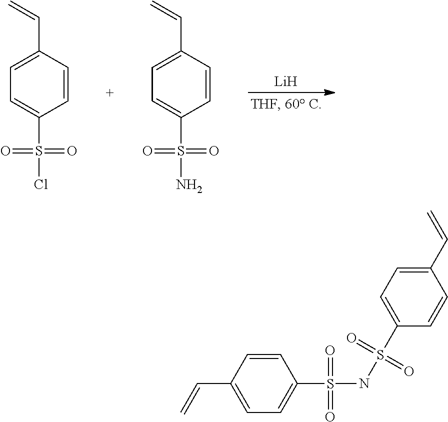

Before the synthesis, benzene sulphonamide was dried in a vacuum oven overnight (30° C., vac). To a solution of the dried benzene sulfonamide (11.12 g, 0.061 mol, 1 moleq) and 4OH-TEMPO (30 mg, 500 ppm) in THF (100 mL) was added LiH (1.06 g, 0.134 mol, 2.2 moleq) as a solid at once. The reaction mixture was stirred for 30 minutes at room temperature. Then, a solution of Cl-SS (12.3 g, 0.061 mol, 1 moleq) in THF (50 mL) was added to the reaction mixture. After addition, the reaction mixture was heated to 60° C. (water bath temperature). After two days, the reaction mixture was filtrated over celite to remove the excess of LiH. Celite was added and the resulting slurry was stirred for 5 minutes. Then, the celite was filtered off and washed with 100 mL ethyl acetate. The solvent was then evaporated in vacuo and the resulting white foam was washed with 500 mL diethyl ether overnight. The resulting white powder was filtered off and dried in a vacuum oven at 30° C. for 16 h yielding a white solid. Typical yield is 11 g (51%). HPLC-MS purity>94%; ¹H-NMR: <1 wt % residual solvents, <5 wt % styrene sulphonate or styrene sulphonamide; ICP-OES: 21-26 g Li/kg product.

### XL-2

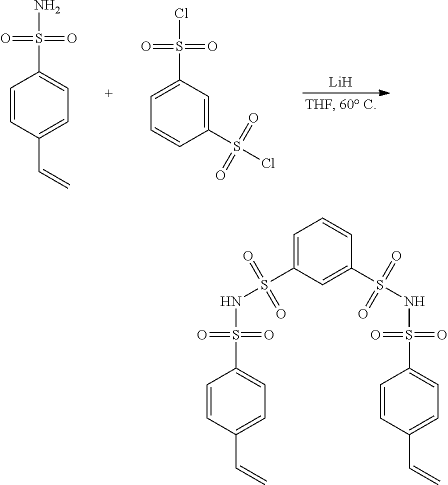

Before the synthesis, styrene sulphonamide was dried in a vacuum oven overnight (30° C., vac). To a solution of the dried styrene sulphonamide (16.90 g, 0.092 mol, 2.05 moleq) and 4OH-TEMPO (30 mg, 500 ppm) in THF (100 mL) was added LiH (1.50 g, 0.189 mol, 4.2 moleq) as a solid at once. The reaction mixture was stirred for 30 minutes at room temperature. Then, a solution of 1,3 benzene disulphonyl chloride (12.38 g, 0.045 mol, 1 moleq) in THF (50 mL) was added to the reaction mixture. After addition, the reaction mixture was heated to 60° C. (water bath temperature). After 2 days, the reaction mixture was filtrated over celite to remove the excess of LiH. The filtrate was concentrated in vacuo to give a light yellow foam. The resulting foam was dissolved in 500 mL ethyl acetate. Celite was added and the resulting slurry was stirred for 5 minutes. Then, the celite was filtered off and washed with 100 mL ethyl acetate. This Celite procedure was then repeated. The solvent was then evaporated in vacuo and the resulting white foam was washed with 500 mL diethyl ether overnight. The resulting white powder was filtered off and dried in a vacuum oven at 30° C. for 16 h yielding a hygroscopic white solid. Typical achieved yield is 14.5 g (54%). HPLC-MS purity>96%; ¹H-NMR: <2 wt % residual solvents; <2 wt % styrene sulphonamide; ICP-OES: 35-40 g Li/kg product.

### XL-3

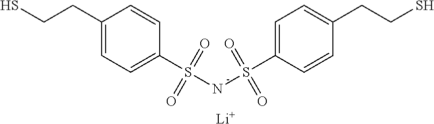 [SMILES: O=S(=O)(NS(=O)(=O)c1ccc(CCS)cc1)c1ccc(CCS)cc1 ; [Li+]]

In 100 mL four-necked flask quaternary ammonium cellulose triacetate amine base catalyst (synthesized as described in CN104276987 A, 0.8 g) and ethanol (20 mL) were stirred slowly under nitrogen for 30 min. The mixture was then heated to 50° C., and then the reaction solution was bubbled with hydrogen sulphide gas at a rate of 1 Lh. A solution of XL-B (5 g) dissolved in 20 mL of ethanol was added dropwise for a period of 1 h. The solution was stirred an additional hour at 50° C. while continuously bubbling hydrogen sulphide gas. Then the gas flow was stopped and the reaction was allowed to room temperature. The reaction crude was filtered off and the solvent was evaporated to yield XL-3 (yield 85%). HPLC-MS purity>97%; ¹H-NMR: <2 wt % residual solvents; <2 wt % XL-B ; ICP-OES: 19-22 g Li/kg product.

Preparation of Component (iii)

MM-P and MM-M (referred to above) had the structures shown below.

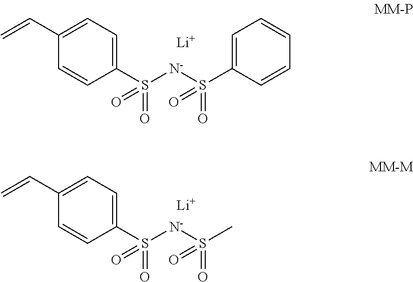 [SMILES: C=Cc1ccc(S(=O)(=O)[N-]S(=O)(=O)c2ccccc2)cc1 ; [Li+] ; C=Cc1ccc(S(=O)(=O)[N-]S(C)(=O)=O)cc1 ; [Li+]]

The compounds MM-P and MM-M were synthesized according to the following general scheme and procedure:

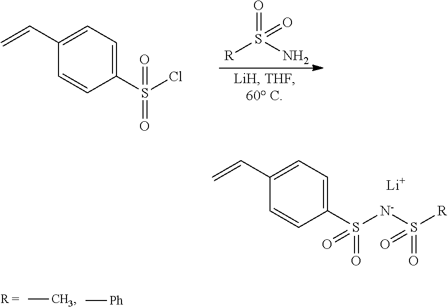

### General Procedure

Before the synthesis, the corresponding sulphamide was dried in a vacuum oven overnight at 30° C. To a solution of the dried sulphamide (0.100 mol, 1 moleq) and 4OH-TEMPO (30 mg, 500 ppm) in THF (100 mL) was added LiH (0.300 mol, 3 moleq) as a solid at once. The reaction mixture was stirred for 30 minutes at room temperature. Then, a solution of vinyl benzyl sulphonyl chloride (0.100 mol, 1 moleq) in THF (50 mL) was added and the reaction mixture was heated to 60° C. (water bath temperature) for 16 h. The resulting solution was filtrated over celite and the resulting foam was dissolved in 500 mL ethyl acetate. Celite was added and the resulting slurry was stirred for 5 minutes. Then, the celite was filtered off and washed with 100 mL ethyl acetate. The solvent was then evaporated in vacuum and the resulting white foam was crushed with 500 mL diethyl ether overnight. The resultant compound of Formula (IV) was collected by filtration and isolated as a white hygroscopic powder. Data on yield and purity are given in Table 3 below.

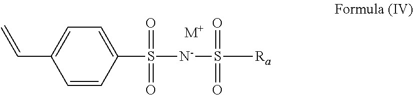

TABLE 3
Compounds of Formula (IV)

| | | | Residual | Residual | |
|---|---|---|---|---|---|
| Rₐ | Yield | Purity | solvent | LiSS | Li content |
| methyl | 80% | >94% | <1% | <4% | 26-30 g/kg |
| phenyl | 79% | >96% | <1% | <2% | 23-28 g/kg |

### Preparation and Testing of the Examples

Compositions were prepared comprising the ingredients shown in Table 4 below in the amounts shown (in wt %).

Polymer films (cation exchange membranes) according to the first aspect of the present invention and Comparative Examples were prepared by applying each of the compositions described in Table 4 onto a FO2223-10 porous support from Freudenberg Filtration Technologies using a 100 μm Mayer bar and then curing the composition by one or more of the methods indicated in Table 4. UV curing was performed by placing the samples on a conveyor at 5 m/min equipped with a D and a H-bulb or 385 nm LED and exposing the wet coatings to either one or both of them, depending on the photoinitiator system. In all cases, UV-curing or EB curing was applied as the first curing step and thermal curing or EB curing as second curing step. EB curing was performed by placing the samples in a conveyor and flushing the whole system with nitrogen. An electron beam of 200 KeV was applied. The dose was varied by adjusting the conveyor speed. A dose of 100 kGy was applied when EB was selected as first curing step. The dose was reduced when EB was used as a second curing step. 80 kGy were applied for samples containing XL-D or XL-B whereas 40 to 60 kGy were used for samples containing XL-2. Thermal curing was performed by placing the polymer film samples, packed in a sealed plastic bag, in an oven set at 90° C. for 10 h. This formed a polymer film (including the porous support) of thickness 100 μm. After the preparation of the polymer films they were stored in sealed aluminum bags and stored at room temperature (20° C.) prior to further analysis.

In table 4, the curing methods applied are specified per example.

The PS, ER, swelling and content of components (a) and (b) of the resultant polymer films were measured as described above and the results are shown in Table 4 below.

TABLE 4

| Materials/amounts (wt %) | CEx. 1 | CEx. 2 | CEx. 3 | CEx. 4 | CEx. 5 | CEx. 6 | CEx. 7 |
|---|---|---|---|---|---|---|---|
| MM-M | 25 | 25 | 35 | 25 | 12 | 7 | 7 |
| MM-P | | | | | | | |
| XL-B | | | 25 | | | | |
| XL-D | | | | | 54 | | |
| XL-2 | 35 | 35 | | 35 | | 59 | 59 |
| water | 39 | 33 | 26 | 29 | 18 | 18 | 18 |
| MeOH | | | | | | | |
| IPA | | 6 | 4 | | | | |
| DMSO | | | 9 | | | | |
| NMP | | | | 10 | 13 | | |
| DVB | | | | | | 13 | 13 |
| LAP | 1 | 1 | 1 | 1 | 1 | | 1 |
| Irgacure ™ 1173 | | | | | | | |
| TPO-L | | | | | | | |
| DBzO | | | | | | | |
| V-601 | | | | | | | |
| V-40 | | | | | | | |
| AV-044 | | | | | | | |
| UV curing | X | X | X | X | X | | X |
| Thermal curing | | | | | | | |
| EB curing | | | | | | X | |
| Permselectivity (NaOH) | 31 | 41 | −9 | 48 | 42 | 84 | 75 |
| ER | 1.0 | 1.2 | 0.8 | 1.3 | 1.3 | 1.7 | 1.6 |
| Swelling (%) | 180 | 115 | 245 | 96 | 98 | 35 | 43 |
| Component (a) (mg/g) | >55 | >55 | >55 | >55 | >55 | 39.4 | 47.4 |
| Component (b) (mg/g) | | | | | | 29.0 | 30.9 |

| materials/amounts (wt %) | Ex. 1 | Ex. 2 | CEx. 8 | Ex. 3 | Ex. 4 | CEx. 9 | Ex. 5 |
|---|---|---|---|---|---|---|---|
| MM-M | 7 | 7 | 7 | 7 | 7 | 7 | 7 |
| MM-P | | | | | | | |
| XL-B | | | | | | 59 | 59 |
| XL-D | | | 59 | 59 | 59 | | |
| XL-2 | 59 | 59 | | | | | |
| water | 18 | 18 | 18 | 18 | 18 | 18 | 18 |
| MeOH | | | | | | | |
| IPA | | | | | | | |
| DMSO | | | | | | | |
| NMP | | | | | | | |
| DVB | 13 | 13 | 13 | 13 | 13 | 13 | 13 |
| LAP | 1 | 1 | 0 | 1 | 1 | 1 | 1 |
| Irgacure ™ 1173 | | | | | | | |
| TPO-L | | | | | | | |
| DBzO | 0.5 | | | | 0.5 | | |
| V-601 | | | | | | | |
| V-40 | | | | | | | |
| AV-044 | | | | | | | |
| UV curing | X | X | X | X | X | X | X |
| Thermal curing | X | | | | X | | |
| EB curing | | X | | X | | | X |
| Permselectivity (NaOH) | 93 | 92 | 56 | 87 | 85 | 69 | 92 |
| ER | 2.7 | 2.7 | 1.2 | 2.6 | 2.5 | 2.2 | 2.8 |
| Swelling (%) | 30 | 28 | 56 | 31 | 34 | 43 | 29 |
| Component (a) (mg/g) | 5.6 | 5.9 | 42.2 | 6.2 | 6.6 | 37.4 | 5.0 |
| Component (b) (mg/g) | 2.4 | 2.8 | 30.1 | 3.2 | 3.4 | 25.7 | 2.1 |

| materials/amounts (wt %) | Ex. 6 | Ex. 7 | Ex. 8 | Ex. 9 | Ex. 10 | Ex. 11 | Ex. 12 |
|---|---|---|---|---|---|---|---|
| MM-M | 7 | | | | | | |
| MM-P | | 13 | 13 | 13 | 12.6 | 12.7 | 12.8 |
| XL-B | 59 | 54 | 54 | 54 | 54.2 | 54.9 | 55.6 |
| XL-D | | | | | | | |
| XL-2 | | | | | | | |
| water | 18 | 16.5 | 16.5 | 16.5 | 17.8 | 17.6 | 17.4 |
| MeOH | | | | | 2 | 4 | 6 |
| IPA | | | | | | | |
| DMSO | | | | | | | |
| NMP | | | | | | | |
| DVB | 13 | 13 | 13 | 13 | 10.4 | 7.8 | 5.2 |
| LAP | 1 | | | | 0.5 | 0.5 | 0.5 |
| Irgacure ™ 1173 | | 0.5 | 0.5 | 0.5 | | | |
| TPO-L | | 0.5 | 0.5 | 0.5 | 0.5 | 0.5 | 0.5 |
| DBzO | 0.5 | | | | | | |
| V-601 | | | 1 | | | | |
| V-40 | | 1 | | | 1 | 1 | 1 |
| AV-044 | | | | 1 | | | |
| UV curing | X | X | X | X | X | X | X |
| Thermal curing | X | X | X | X | X | X | X |
| EB curing | | | | | | | |
| Permselectivity (NaOH) | 96 | 99 | 98 | 98 | 97 | 97 | 96 |
| ER | 5.2 | 6.2 | 5.9 | 5.8 | 5.6 | 4.9 | 4.2 |
| Swelling (%) | 24 | 19 | 21 | 22 | 23 | 22 | 23 |
| Component (a) (mg/g) | 2.5 | 1.6 | 2.0 | 1.8 | 2.2 | 2.0 | 2.2 |
| Component (b) (mg/g) | 3.1 | 2.4 | 2.7 | 2.9 | 3.0 | 2.8 | 2.7 |

| materials/amounts (wt %) | Ex. 13 | CEx. 10 | Ex. 14 | Ex. 15 | Ex. 16 | Ex. 17 |
|---|---|---|---|---|---|---|
| MM-M | | | | | | |
| MM-P | 12.9 | 13 | 12.2 | 19.6 | 13 | 13 |
| XL-B | 56.3 | 57 | 52.5 | 45.1 | | |
| XL-D | | | | | | |
| XL-E | | | | | 54 | |
| XL-P | | | | | | 54 |
| XL-2 | | | | | | |
| water | 17.2 | 17 | 19.5 | 19.5 | 17.0 | 17.0 |
| MeOH | 8 | 10 | | | | |
| IPA | | | | | | |
| DMSO | | | | | | |
| NMP | | | | | | |
| DVB | 2.6 | 0 | 14.3 | 14.3 | 13 | 13 |
| LAP | 0.5 | 0.5 | | | 0.5 | 0.5 |
| Irgacure ™ 1173 | | | 0.5 | 0.5 | | |
| TPO-L | 0.5 | 0.5 | 0.5 | 0.5 | 0.5 | 0.5 |
| DBzO | | | | | | |
| V-601 | | | | | | |
| V-40 | 1 | 1 | 0.5 | 0.5 | 0.5 | 0.5 |
| AV-044 | | | | | | |
| UV curing | X | X | X | X | X | X |
| Thermal curing | X | X | X | X | X | X |
| EB curing | | | | | | |
| Permselectivity (NaOH) | 95 | 82 | 96 | 93 | 95 | 87 |
| ER | 3.7 | 1.8 | 5.6 | 5.2 | 4.9 | 3.3 |
| Swelling (%) | 25 | 36 | 23 | 26 | 25 | 27 |
| Component (a) (mg/g) | 6.0 | 16.7 | 5.3 | 4.0 | 3.2 | 2.9 |
| Component (b) (mg/g) | 3.2 | | 3.1 | 3.6 | 3.2 | 3.4 |

TABLE 5
Further Examples (using a component (b) other than DVB)

| materials/amounts (wt %) | Ex. 18 | Ex. 19 | Ex. 20 | Ex 21 | Ex. 22 | Ex. 23 | Ex 24 |
|---|---|---|---|---|---|---|---|
| water | 18.35 | 18.35 | 18.35 | 18.5 | 18.5 | 18.5 | 18.5 |
| TAOT | 13.15 | | | 15 | | | |
| PETAE | | 13.15 | | | 15 | | |
| TVCH | | | 13.15 | | | 15 | |
| TCA | | | | | | | 14 |
| LAP | 1 | 1 | 1 | 1 | 1 | 1 | 1 |
| OH-TEMPO (2% in PW) | 1 | 1 | 1 | 1 | 1 | 1 | 1 |
| MM-M | 12.25 | 12.25 | 12.25 | | | | |
| XL-D | 53.75 | 53.75 | 53.75 | | | | 65 |
| XL-3 | | | | 64 | 64 | 64 | |
| DBzO | 0.5 | 0.5 | 0.5 | 0.5 | 0.5 | 0.5 | 0.5 |
| UV curing | X | X | X | X | X | X | X |
| Thermal curing | X | X | X | X | X | X | X |
| Permselectivity | 90 | 85 | 86 | 90 | 85 | 86 | 91 |
| ER | 2.6 | 1.9 | 2.1 | 2.6 | 1.9 | 2.1 | 2.7 |
| Swelling (%) | 27 | 31 | 28 | 27 | 31 | 28 | 28 |
| Component (a) (mg/g) | 13.9 | 14.5 | 14.9 | 9.2 | 8.3 | 10.1 | 9.4 |
| Component (b) (mg/g) | 5.0 | 4.6 | 4.2 | 2.6 | 2.0 | 2.8 | 1.7 |

Comparative examples 1 to 5 and 10 do not contain component (b) and as a result thereof have a low permselectivity.

Comparative examples 6, 7, 8 and 9 are prepared with only one curing step and as a result thereof contain too high an amount of components (a) and/or (b).

1. A polymer film comprising anionic groups and 0.008 to 25 mg/g of each of components (a) and (b):
(a) a crosslinking agent which is free from fluoro groups and comprises a group of Formula (I); and
(b) a non-ionic crosslinking agent of Formula (III); 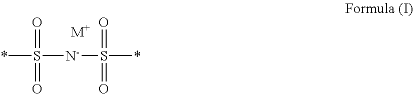
wherein M⁺ is a cation and * indicates the attachment points to other elements of the crosslinking agent;
R′ₙ-A Formula (III)
wherein:
each R′ independently comprises a polymerisable group or a non-polymerisable group;
n has a value of 2, 3 or 4; and
A is a linking group;
provided that the compound of Formula (III) comprises at least two polymerisable groups and is free from ionic groups.

2. The polymer film according to claim 1 wherein component (a) is of Formula (II):
wherein: 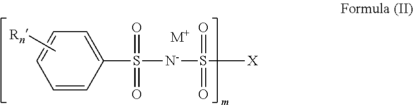
n′ has a value of 1 or 2;
m has a value of 1 or 2;
M⁺ is a cation;
each R independently is a polymerisable or non-polymerisable group; and
X is an optionally substituted amine group, an optionally substituted alkylene group or an optionally substituted arylene group;
provided that the compound of Formula (II) comprises at least two polymerisable groups.

3. (canceled)

4. The polymer film according to claim 1 wherein:
(i) n has a value of 2, 3 or 4 and A is C₁₋₆-alkylene or C₆₋₁₈-arylene; or
(ii) A is N, n has a value of 3 and either all three of the groups represented by R′ comprise a polymerisable group or two of the groups represented by R′ comprise a polymerisable group and the third group represented by R′ is H or C₁₋₄ alkyl; or
(iii) A is a triazine group or a cyanuric acid derivative, n has a value of 3 and either all three of the groups represented by R′ comprise a polymerisable group or two of the groups represented by R′ comprise a polymerisable group and the third group represented by R′ is C₁₋₄ alkyl or C₁₋₄ alkoxy.

5. The polymer film according to claim 2 wherein m and n′ both have a value of 1 and X is a phenylene group carrying a vinyl group.

6. The polymer film according to claim 2 wherein m has a value of 2, X is a C₁₋₆-alkylene or C₆₋₁₈-arylene group, or X is a group of the formula N(R″)₍₃₋ₘ₎ wherein each R″ independently is H or C₁₋₄-alkyl.

7. The polymer film according to claim 2 wherein m has a value of 1, n′ has a value of 2 and X is a C₁₋₆-alkyl, a C₆₋₁₈-aryl group or a group of the formula N(R″)₂ wherein each R″ independently is H or C₁₋₄-alkyl.

8. The polymer film according to claim 1 wherein the polymerisable groups each independently comprise an ethylenically unsaturated group, an epoxide group or a thiol group.

9. The polymer film according to claim 1 wherein component (a) comprises polymerisable groups which are copolymerisable with polymerisable groups present in component (b).

10. The polymer film according to claim 1 wherein component (b) comprises divinylbenzene, 2,4,6-triallyloxy-1,3,5-triazine, 1,3,5-triallylisocyanurate, triallylamine, 1,2,4-trivinylcyclohexane, tetra(allyloxy)ethane, pentaerythritol tetraallyl ether, 2,3-dimercapto-1-propanol, dithioerythritol, or trithiocyanuric acid or a combination thereof.

11. A composition comprising:
(i) component (a) as defined in claim 1;
(ii) component (b) as defined in claim 1;
optionally (iii) a compound comprising one and only one polymerisable group;
optionally (iv) one or more radical initiators; and
optionally (v) a solvent.

12. A polymer film obtainable by curing a composition according to claim 11.

13. A process for preparing a polymer film comprising the steps of:
i. providing a porous support;
ii. impregnating the porous support with a composition according to claim 11, wherein the composition comprises 0.008 to 25 mg/g of each of components (a) and (b); and
iii. curing the composition.

14. The process according to claim 13 wherein the curing comprises a first curing step and a second curing step.

15. The process according to claim 14 wherein the first and second curing steps are respectively selected from (i) UV curing then thermal curing; (ii) UV curing then electron beam curing; and (iii) electron beam curing then thermal curing.

16. The process according to claim 14 which comprises curing the composition in the first curing step to form a polymer film, winding the polymer film onto a core, optionally together with an inert polymer foil, and then performing the second curing step, or curing the composition in the first curing step, performing the second curing step and winding the polymer film onto a core, optionally together with an inert polymer foil.

17. A cation exchange membrane comprising the polymer film according to claim 1.

18. A method of using the cation exchange membrane according to claim 17 for the treatment of a polar liquid or for the generation of electricity.

19. The polymer film according to claim 1 wherein the polymerisable groups are selected from the group consisting of vinyl groups, epoxide groups and thiol groups.

20. The polymer film according to claim 9 wherein the polymerisable groups are selected from the group consisting of vinyl groups, epoxide groups and thiol groups.
